(19) United States
(12) Patent Application Publication
(10) Pub. No.: US 2007/0239151 A1
(43) Pub. Date: Oct. 11, 2007

(54) METHOD AND APPARATUS FOR THE DETACHMENT OF CATHETERS OR PUNCTURING OF MEMBRANES AND INTRALUMINAL DEVICES WITHIN THE BODY

(75) Inventors: Ergin Atalar, Ankara (TR); Anil Arat, Houston, TX (US); Tonguc Onur Tasci, Salt Lake City, UT (US)

Correspondence Address: CONLEY ROSE, P.C.;David A. Rose, P. O. BOX 3267, HOUSTON, TX, 77253-3267, US

(73) Assignee: BAYLOR COLLEGE OF MEDICINE, Houston, TX (US)

(21) Appl. No.: 11/697,239

(22) Filed: Apr. 5, 2007

Related U.S. Application Data
(60) Provisional application No. 60/789,782, filed on Apr. 6, 2006.

(51) Int. Cl. A61B 18/04

(52) U.S. Cl. 606/27

(57) ABSTRACT

The disclosed methods and devices utilize various techniques to detach the distal end of a catheter from an obstruction with minimal invasiveness and effort by the surgeon. As reflux of an embolic agent or hardening material over the catheter tip is a major causative factor in the increased morbidity/mortality of embolization procedures and also a technical limitation preventing a better cure rate, a method has been developed for the detachment of the distal end of catheters within the body, preferably with no regard to the amount of reflux, and preferably at the proximal edge of the reflux, in order to be able to make embolization procedures safer and more effective.

### CROSS-REFERENCE TO RELATED APPLICATIONS

This application claims priority to U.S. provisional application Ser. No. 60/789,782, filed on Apr. 6, 2006 and incorporated herein by reference in its entirety for all purposes.

### BACKGROUND

### FIELD OF THE INVENTION

This invention relates generally to the field of catheters. More specifically, the invention relates to a method and apparatus for the treatment of vascular malformations, aneurysms, tumors, or hemorrhages.

### BACKGROUND OF THE INVENTION

Typical treatment of arteriovenous malformations (AVMs) involves endovascular treatment, surgery and radiotherapy. An AVM is a congenital disorder of the blood vessels in the brain, characterized by tangles of veins and arteries that lack the normal capillary structure. Currently the standard endovascular treatment involves obliteration of the malformation or the fistula with embolic agents such as cyanoacrylic glue, other proprietary materials (i.e. Onyx™) or in some cases by particles such as polyvinyl alcohol (PVA). Interventional treatment of tumors also involves the same endovascular tools.

The chance for an endovascular cure with cyanoacrylates only is not high. This is mainly because of the technical difficulties related to the use of acrylic glue. This entails expertise, attention and adherence to a strict technique which was developed over the years to prevent either inadvertent embolization or gluing of the catheter to brain vessels. Among these two complications, gluing of the catheter tip is a well-recognized complication that may be distressing. In several series, this complication has been reported in up to 10% of procedures, sometimes with serious outcomes. The scarcity of literature data may as well be secondary to under-reporting of this complication with an unknown actual risk of permanent catheter fixation.

When gluing of the catheter tip occurs, there are two methods for salvage. The first one is to leave the catheter in place, which is extending from the embolized lesion to the groin (access site). Although there are case reports documenting the incorporation of the retained catheters into the cerebral vasculature, it is also stated that epithelization does not occur quickly increasing the risk of thromboembolic events. The patients with the retained catheters need to be followed under anticoagulation or antiplatelet therapy which is not preferred in patients with cerebral vascular malformations. The number of embolizations that can be performed via the same vascular pedicle is also limited with this approach, as with each subsequent embolization, there will be the risk of retaining more than one catheter in intracranial arteries. The outcome of this approach is currently not well-known.

The second method is to severe the catheter at its distal portion by pulling with a sudden thrust and leaving the distal fragment of the catheter in the cerebral vasculature. Despite the allegations of several authors that many patients tolerate this maneuver, major morbidity and mortality has been documented as a result of the performance of this maneuver either secondary to vascular avulsion/intracranial bleeding or to inadvertent embolization of polymerized glue by an adherent droplet being shorn from the tip of the microcatheter and as such, catheter fixation remains a highly undesirable event among endoneurovascular operators. Surgical removal of these catheters may sometimes be needed.

The most important factor in preventing catheter adhesion is limitation or prevention of reflux along the microcatheter This not only requires considerable endovascular skills and expertise, but also limits the success of the embolization procedure. The penetration of the embolic agent into the target site is enhanced by the formation of an “intravascular plug” at the catheter tip. Generally, however, the formation of this plug necessitates a small reflux of the embolic agent along the catheter tip.

Consequently, there is a need for a device which allows a surgeon to easily detach a catheter tip during treatment of AVMs.

### BRIEF SUMMARY

The disclosed methods and devices utilize various techniques to detach the distal end of a catheter from an obstruction with minimal invasiveness and effort by the surgeon. As reflux of an embolic agent or hardening material over the catheter tip is a major causative factor in the increased morbidity/mortality of embolization procedures and also a technical limitation preventing a better cure rate, a method has been developed for the detachment of the distal end of catheters within the body, preferably with no regard to the amount of reflux, and preferably at the proximal edge of the reflux, in order to be able to make embolization procedures safer and more effective.

These and other needs in the art are addressed in one embodiment by a device comprising a catheter having an open distal end, wherein said catheter comprises a material having a melting point. The device also includes a heating element disposed at the distal end of said catheter, where the heating element causes the distal end to detach by the heating of said distal end to at least the melting point of said material.

In an embodiment a device comprises a catheter having an open distal end. The device also comprises a support member disposed parallel to the catheter. The device further comprises a means of detaching said open distal end from said catheter coupled to the support member. The means of detaching said open distal end form said catheter may be a mechanical means or a chemical means.

In yet another embodiment, a method comprises inserting a catheter having an open distal end. The method additionally comprises injecting a hardening material through the open distal end to form an obstruction. Moreover, the method comprises positioning a heating element at the open distal end. The method also comprises heating at least a portion of the open distal end using the heating element so as to detach the catheter from the open distal end and the obstruction.

According to an embodiment, a method comprises inserting a catheter having an open distal end. The method additionally comprises injecting a hardening material through the open distal end to form an obstruction. The method further comprises positioning a means for detaching the catheter from the obstruction at the distal end. The means of detaching said open distal end form said catheter may be a mechanical means or a chemical means. In addition, the method comprises using the mechanical or chemical means to detach the catheter from the obstruction.

The foregoing has outlined the features and technical advantages of the present invention in order that the detailed description of the invention that follows may be better understood. Additional features and advantages of the invention will be described hereinafter that form various embodiments of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a detailed description of the embodiments of the invention, reference will now be made to the accompanying drawings in which:

FIGS. 1(a)-(c) illustrates various embodiments of a device with a heating element;

FIG. 2 illustrates an embodiment of a device with a heating element; and

FIGS. 3A-B illustrates an embodiment of a device with a mechanical means of removing the catheter from an obstruction;

FIG. 4 illustrate an embodiment of a device with a mechanical means of removing the catheter from an obstruction;

FIG. 5 illustrates an embodiment of a device with an inner mechanical means of removing the catheter from an obstruction;

FIG. 6 illustrates an embodiment of a device with an optical source for detaching distal end of a catheter from an obstruction;

FIG. 7 illustrates an embodiment of a device utilizing a chemical substance to remove distal end of a catheter from an obstruction;

FIG. 8 illustrates an experimental setup for testing embodiments of the device; and

FIG. 9 is a plot of temperature versus time at the distal end of the device.

### Notation and Nomenclature

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct electrical connection. Thus, if a first device couples to a second device, that connection may be through a direct electrical connection, or through an indirect electrical connection via other devices and connections.

### DETAILED DESCRIPTION OF THE PREFERRED EMBODIMENTS

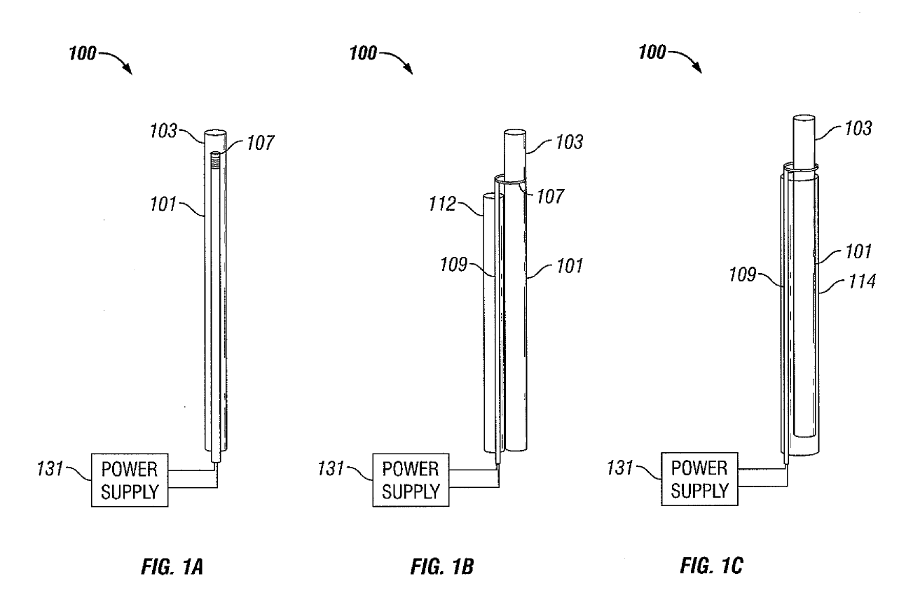

FIG. 1 illustrates various embodiments of a vascular device for treating vascular abnormalities such as AVMs, aneurysms, tumor vessels or bleeding sites. In an embodiment, the device 100 comprises a catheter 101 and an open distal end 103. As defined herein, a catheter is any conduit or hollow body that may be inserted into the vasculature of a patient. Typically, catheter 101 is inserted over a guide wire (not shown). However, it is contemplated that other embodiments of the device 100 do not require use of a guide wire. The catheter 101 may be made of any suitable biocompatible material. Examples of suitable materials include plastics, copolymers, alloys, metals and the like. Examples of polymers or plastics that the catheter may be made include without limitation, polyethylene, polypropylene, polyurethane, silicone rubber, and the like. Such materials traditionally have a relatively low melting point. As used herein, melting point is the temperature at which a material begins to change from solid to liquid. In addition, device 100 may comprise any catheter 101 that is commercially available and currently used by those of skill in the art.

In a further embodiment, device 100 comprises a heating element 107 disposed at distal end of catheter 101. According to an embodiment, heating element 107 is disposed within distal end 103 of catheter 101 as shown in FIG. 1(a). Alternatively, heating element 107 may be disposed circumferentially around distal end as shown in FIGS. 1(b)-(c) to form a heating lasso or loop. Heating element 107 serves to detach distal end 103 from catheter 101 by site-specific melting of catheter 101 near heating element 107, thereby detaching distal end 103 from catheter 101.

Heating element 107 is typically coupled to a power source 131 via a support member 109 which is preferably longitudinally coaxial or parallel to catheter 101. Typically, support member 109 is a wire which provides power to heating element 107. However, support member 109 may be any structure that allows positioning of heating element 107 to the desired location of the catheter 101. Depending on the embodiment, support member 109 may be disposed external to catheter 101 as shown in FIG. 1(a) or may be disposed within catheter 101 as in FIGS. 1(b)-(c). In embodiments where support member 109 is disposed externally to catheter 101, support member 109 may be run through an auxiliary catheter 112. Auxiliary catheter 112 may be positioned parallel to catheter 101. Alternatively, an outer or guiding catheter 114 may be disposed coaxially around catheter 101, support member 109, and heating element 107. In an embodiment, support member 109 comprises a waveguide to pass a beam of light to heating element 107. In other embodiments, support member 109 comprises a conductive metal such as without limitation, platinum, gold, silver, copper, or combinations thereof

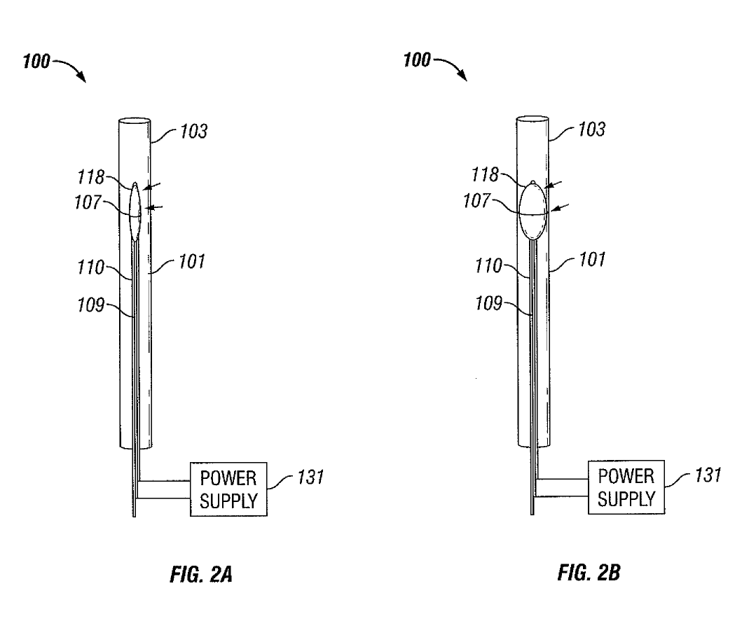

Referring now to FIG. 2, heating element 107 may be attached to the balloon portion 118 of a balloon catheter 110. In this embodiment, balloon catheter 110 is disposed coaxially within catheter 101. When balloon portion 118 of balloon catheter 110 is expanded, heating element 107 may be brought into close proximity with the catheter wall. When balloon portion 118 is deflated, heating element 107 may also contract. When heating element 107 is activated in balloon catheter's expanded state, the increase in temperature causes detachment of distal end 103 from catheter 101 by melting. Preferably, balloon portion 118 is made of a heat-resistant material such that as heating element 107 heats catheter 101, balloon portion 118 remains intact. Any suitable heat-resistant materials known to those of skill in the art may be used.

Heating element 107 may comprise any number of devices known to cause an increase in temperature. Examples of such devices including high resistance coils, lasers, radiofrequency emitter, microwave devices, ultrasound devices, etc. Power is provided to heating element 107 by power supply 131. Power supply 131 may be any apparatus known to those of skill in the art that provides power. Examples include without limitation, batteries, DC power unit, generators, solar power, AC power supplies, or combinations thereof.

In an embodiment, device 100 includes a temperature probe disposed at distal end 103 of catheter 101. Temperature probe may be any device used to monitor temperature or provide temperature information. The temperature probe may be coupled to power supply 131 to provide a feedback loop for heating element 107. The feedback loop serves to prevent power supply 131 from overheating element 107 and causing damage to the vasculature.

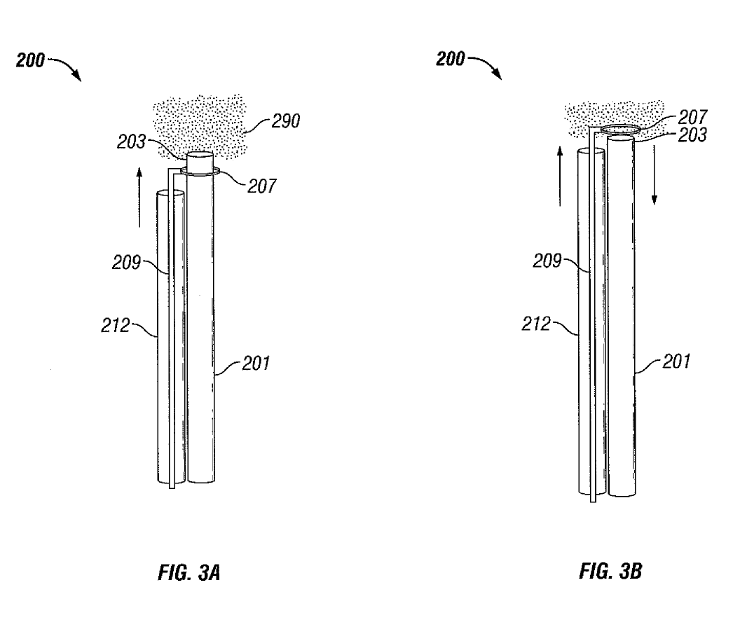

As illustrated in FIGS. 3-5 and 6, embodiments of the device may comprise a means for detaching the distal end of catheter from an obstruction or embolism. The means for detaching the distal end of catheter from an obstruction or embolism may be a chemical means or a mechanical means. As used herein, mechanical means refers to any means using physical force (i.e. cutting, friction) to detach distal end of catheter. On the other hand, chemical means refers to any means of using a chemical substance or fluid to dissolve the catheter wall to detach distal end of catheter.

Referring now to FIG. 3, according to an embodiment, catheter 201 may include an elongate support member 209 running longitudinally along catheter body 201. Elongate member 209 typically comprises a wire. Furthermore, mechanical means may comprise a loop 207 surrounding distal end of catheter. In an embodiment, loop 207 is coupled to elongate support member 209. Elongate member 209 serves to mechanically move loop longitudinally along catheter 201. As described above, elongate member 209 may also be disposed within auxiliary catheter 212.

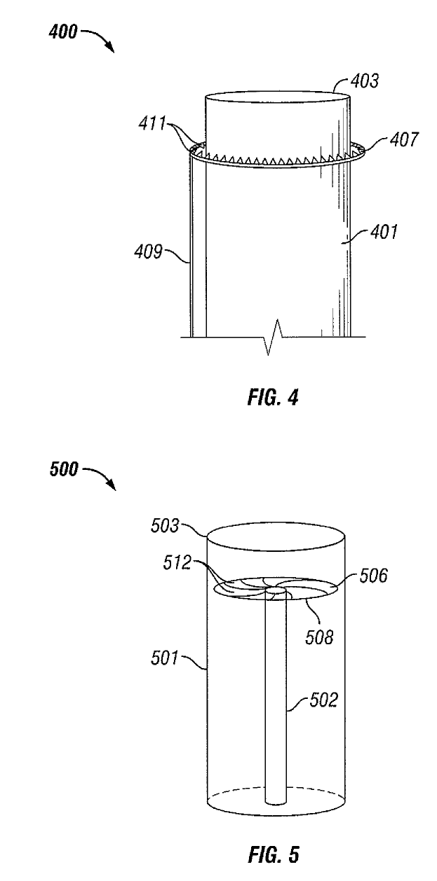

Now referring to FIG. 4, in an embodiment, loop 407 or lasso may comprise a plurality of cutting elements 411 such as without limitation, teeth or blades. Loop 407 may additionally include a noose or other mechanism to tighten loop around catheter. As loop 407 is tightened, plurality of cutting elements 411 engage catheter 401 and cut distal end 403 from catheter 401. That is, the tightening of loop 407 serves to detach distal end 403 from catheter 401.

In an embodiment, mechanical means 507 may be disposed within catheter 501 as shown in FIG. 5. For example, mechanical means 507 may comprises an inner cutting element 506 that is coupled to an inner support member 502. In one aspect, inner support member 502 comprises an inner catheter. According to one embodiment, inner cutting element 506 may be expandable i.e. having an expanded position and a collapsed position. In particular, inner cutting element 506 may comprise an expandable disk. Moreover, inner cutting element 506 may have sharpened or serrated edges 508 to cut through the catheter wall. In an embodiment, inner cutting element 506 comprises a plurality of overlapping leaves 512 which contract and expand radially. Alternatively, inner cutting element 506 may comprise an umbrella-type configuration in which inner cutting element 506 collapses and folds against inner support member 502.

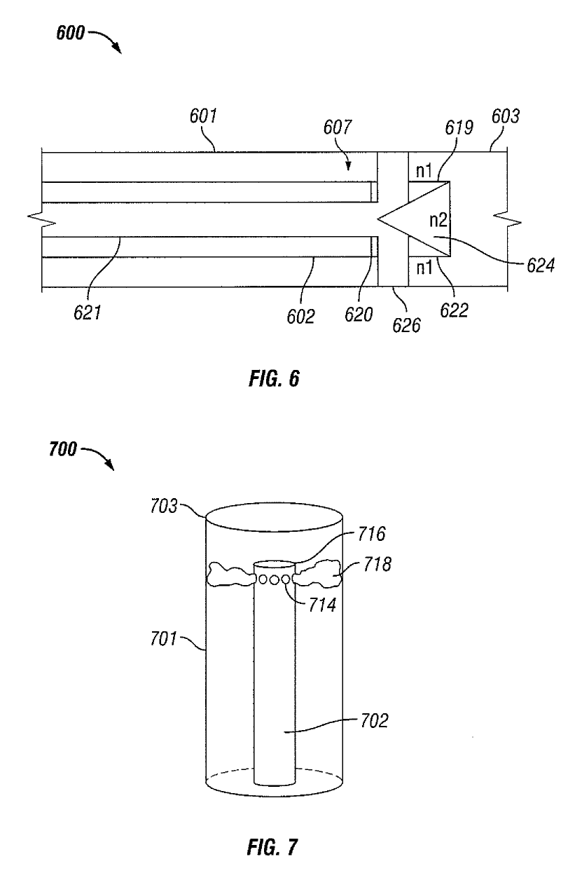

Referring to FIG. 6, in yet another embodiment, the device may utilize a laser to detach the distal end 603 of catheter 601. A laser beam 621 may be directed down a waveguide 602 which is inserted coaxially into catheter 601. According to an embodiment, waveguide 602 comprises a fiber optic cable. Distal end 619 of waveguide 602 may also comprise a beam splitter 620. Splitter 620 may split beam 621 into a plurality of beams directed at the catheter 601 wall. Furthermore, splitter 620 may comprise at least two different glass elements 622, 624 with refractive indices n₁ and n₂ , respectively, may be disposed at the distal end 619 of waveguide 602. Glass element 624 may have a conical shape and a tip angle, α, in which α is selected so that the incoming laser beam 621 is directed to the inner surface of catheter 601. The re-directed beams heat the inner surface of catheter 601 so as to melt the catheter wall and detach distal end 619.

According to an embodiment, device 700 comprises an inner catheter 702 having a closed distal tip 716 (See FIG. 7). Distal tip 716 of the inner catheter 702 may comprise a plurality of openings 714 on its outer surface. In an embodiment, the plurality of openings 714 are arranged circumferentially around inner catheter 702 at the distal end 716 of catheter 702 in a band. However, openings 714 may be arranged in any configuration which may optimally allow ejection of a chemical substance for detachment of distal end 703 of catheter 701. Distal end 716 is generally closed so as to allow ejection of fluid from openings 714 to inner surface of catheter 701.

In an embodiment of a method, the above disclosed device 100 is inserted through the vasculature to the site of the AVM or other vascular abnormality. The device 100 may be inserted with the assistance of a guide wire. In addition, the method may comprise injecting a hardening material (i.e. an embolic agent) through catheter 101 to the AVM to form an obstruction. The obstruction prevents blood from reaching the AVM.

Any suitable material known to those of skill in the art may be used. Examples of suitable materials include without limitation, cyanoacrylate glue, acrylic glue, fibrin glue, adhesives, hydrogels, polymers, or combinations thereof. Recently a commercial precipitating agent or hardening material, Onyx™, has been approved for the endovascular treatment of AVMs. Since this agent is non-adhesive, it permits prolonged injections for embolization. It has become apparent that using this agent with its fall potential entails an intentional reflux of this material at the catheter tip to be able to form an “intravascular plug.” This permits a higher rate of penetration to AVMs with a higher obliteration rate, at the expense of entrapment (instead of adhesion) of the catheter within the vessels in as much as 10% of the lesions embolized in a randomized controlled trial. A shift from the manufacturing of flow guided catheters to that of over-the-wire catheters was noted on the side of the company producing this embolic agent as the latter maybe recovered better in case entrapment occurs.

As the hardening material sets to form a solid obstruction to the AVM or other vascular deformity, the distal end 103 of catheter 101 may be entrapped by the material. Heating element 107 is then guided either through or over catheter 101 to the AVM site as shown in FIGS. 1(a)-(c). Power may be applied to heating element 107 through support member 109 and power supply 131 to heat heating element 107. The heating element 107 causes an increase in temperature to the melting point of the catheter material, which melts only the portion of catheter 101 in close proximity to heating element 107. Generally, heating element 107 completely melts through catheter wall, completely detaching distal portion 103 from catheter 101. However, in some embodiments, heating element 107 may only melt a portion of catheter 101, causing distal portion 103 to be partially detached from catheter 101. Once heating element 107 has been either completely or partially detached, catheter 101 may then be removed from distal portion 103, leaving distal portion 103 entrapped by the hardening material. As shown in FIGS. 1(a)-(c), heating element 107 may heat or melt catheter from the inside or outside of catheter 101.

In an embodiment, after hardening material is injected and set, a balloon catheter 110 including heating element 107 may be inserted through catheter 101 as shown in FIG. 2. Typically, balloon catheter 110 is inserted with balloon portion 118 in its deflated state. Balloon portion 118 may then be expanded, bringing heating element 107 in close proximity to catheter 101. Voltage or current is applied to heating element 107 to melt the portion of catheter 101 near heating element 107. Any suitable voltages or currents may be applied to heating element 107. Distal end 103 may then be detached from catheter 101 and catheter 101 along with balloon catheter 110 may be withdrawn from the vasculature.

Balloon portion 118 is preferably made of a heat resistant material such that when heating element 107 is heated, balloon portion 118 remains intact and unaffected by the rise in temperatures. An example of such a material includes without limitation, silicone rubber.

As depicted in FIG. 6, in another aspect of the method, after hardening material has set or cured, a heating element 607 utilizing an optical or laser source may be inserted through catheter 601. The distal end of heating element 607 is positioned at the desired location. When properly positioned, a laser beam 621 may be directed down the waveguide 602. Beam splitter 619 divides beam into multiple beams 626 and directs these beams to inner portion of catheter 601. The plurality of beams 626 heat at least a portion of the catheter wall to either detach or partially detach distal end 603 from catheter 601.

In other embodiments, a chemical or mechanical means may be used in place of heating element 107 to detach or partially detach distal end of catheter. For example, a device 200 as shown in FIGS. 3A-B is inserted into the vasculature. As explained above, hardening material is then injected through catheter 201 to form an obstruction 290 or embolus. If distal tip 203 of catheter 201 is trapped by the hardened obstruction 290, a user may use support member 209 and loop 207 to mechanically force distal end 203 from obstruction 290 as shown in FIG. 3. That is, the force of the support member 209 and loop 207 pushing against the obstruction 290 provide leverage for user to pull catheter 201 from obstruction 290.

In an embodiment, a device 400 with loop 407 which includes a plurality of cutting elements 411 (see FIG. 4) may be used in accordance with the methods described above. After the material hardens, loop 407 may be tightened around distal end 403 of catheter 401 using a wire 409. The plurality of cutting elements disposed on loop 407 then sever or cut distal end 403 from catheter 401, allowing withdrawal of catheter 401 from the vasculature. In some cases, loop 407 may be rotated around catheter 401 to cut into the catheter wall.

In an alternative embodiment, a device 500 as shown in FIG. 5 may be used. After injection and hardening of the hardening material, inner support member 502 coupled to inner cutting element 508 is inserted through catheter 501. The inner cutting element 506 may be in a collapsed state to facilitate insertion to distal end 503 of catheter 501. Once inner cutting element 508 is inserted to its desired position, inner cutting element 506 may be expanded to its expanded position. Inner cutting element 506 may have a diameter which is equal or greater than catheter 101. Inner cutting element 506 may then be rotated to cut or detach distal end 503 from catheter 501.

In another embodiment, a device 700 utilizing a chemical means, as shown in FIG. 7, may be used in conjunction with the method. Once the hardening material has been injected and allowed to cure, an inner catheter 702 may be inserted into catheter 701. A chemical substance 718 may then be injected into inner catheter 702 via a syringe or other device. Chemical substance 718 may be forced through the plurality of openings 714, which may eject the chemical substance in a radial direction toward inner portions of the catheter 701, as shown in FIG. 7. Once ejected, chemical substance 718 may dissolve a portion of the catheter 701 and, thus, either completely or partially detach distal end 703 from catheter 701. Examples of chemical substances 718 include without limitation, solvents, acids, or combinations thereof The chemical substance 718 is preferably biocompatible and non-toxic. Once chemical substance 718 has dissolved at least a portion of the catheter 701, the proximal portion of catheter 701 may then be removed from the hardened material, leaving the distal end 703.

It is envisioned that the above methods and devices will not be limited to embolus applications, but may also be used to detach the balloon portions of a balloon catheter or also puncture intravascular devices such as catheters, stents, stent-grafts, covered stents, or surgical grafts.

To further illustrate various illustrative embodiments of the invention, the following example is provided.

### EXAMPLE

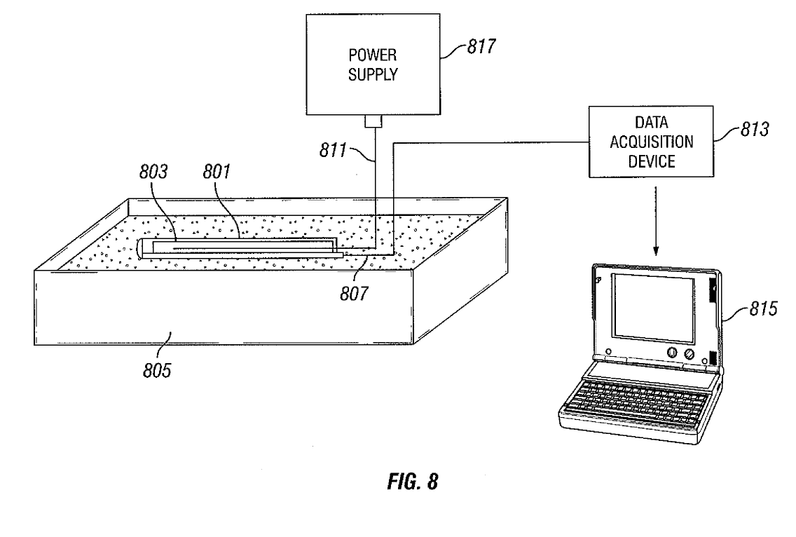

An experimental setup for the testing of the detachment procedure was constructed, which can be seen in FIG. 8. As shown in FIG. 8, a plastic tube 801 was used to simulate an artery, a catheter 803 with non-braided tip and an embolic agent (Onyx® 18 or 50% acrylic glue, Histoacryl® [n-butyl cyanoacrylate in Lipiodol®]) was used and placed in to a water bath 805. The tip of the catheter 803 was positioned within the tube 801 and embolic agent was injected through the catheter to fill the tube. The lumen of the catheter 803 was then flushed with DMSO or D5W for Onyx and glue respectively.

A Micrus-10 coil (with the coil detached) 811 was then advanced into the catheter 803 so that its tip was located within or just adjacent to the embolic cast. A fiber optic temperature probe 807 (FISO Technologies, Ste. Foy, Quebec, and Canada) was inserted near the tip of the coil pusher, which was also connected to the data acquisition device 813 to directly monitor the temperature on the screen of the computer 815. The Micrus-10 wire 811 was connected to a DC switching power supply 817 and voltages from 5 Volts to 15 Volts were applied corresponding to different types of catheters. During the procedure, catheter detachments were visualized by fluoroscopic imaging.

Results

In the experiments, for the embolizations with ONYX, Ultraflow, Baltacci™ 18 and Fastracker® 325 catheters were used and for the embolizations with acrylic glue, Baltacci™, Spinnaker 18 and Ultraflow™ catheters were used. For the experiments, where the coil pusher was within the embolic cast, all the catheters could be detached successfully. On the other hand, no detachment was observed when the tip of the coil pusher was adjacent (outside) the embolic cast. This shows us that the correct placement of the coil pusher tip is crucial (carrying the electrical resistance coil) for a successful catheter detachment. Bubble formation from detachment of the catheters was observed with fluoroscopic imaging.

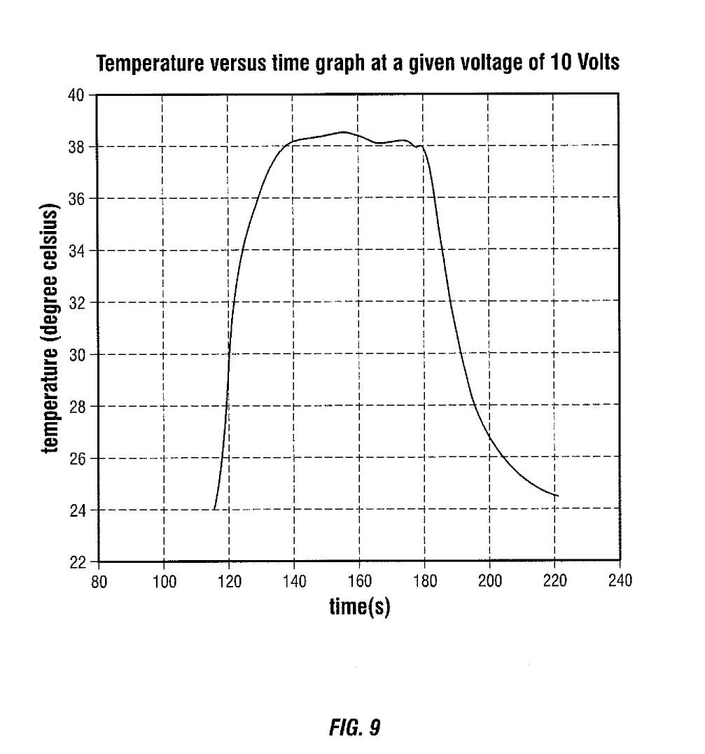

During the experiments the catheter temperature at the detachment region was monitored continuously. Corresponding to the detachment of the Ultraflow catheter with input voltage of 10 volts, the temperature-time graph was observed in FIG. 9. Here, power was given for 25 seconds and detachment was successful. From FIG. 9, one can see that the maximum change in the temperature was around 14 degrees Celsius.

In summary, independent of their brands, all of the catheters with non-braided tips could be detachable by the method that we have explained above. Furthermore this method can be applicable to puncture or ablate intravascular tools like endovascular balloons or stent grafts as well as tissues like vessel wall or membranous tissues.

While embodiments of this invention have been shown and described, modifications thereof can be made by one skilled in the art without departing from the spirit or teaching of this invention. The embodiments described herein are exemplary only and are not limiting. Many variations and modifications of the system and apparatus are possible and are within the scope of the invention. Accordingly, the scope of protection is not limited to the embodiments described herein, but is only limited by the claims which follow, the scope of which shall include all equivalents of the subject matter of the claims.

What is claimed is:

1. A device comprising:
a catheter having an open distal end, wherein said catheter comprises a material having a melting point;
a heating element disposed at the distal end of said catheter, wherein said heating element causes said distal end to detach by the heating of said distal end to at least the melting point of said material.

2. The device of claim 1 wherein said heating element is disposed coaxially within said catheter.

3. The device of claim 1 wherein said heating element is disposed circumferentially around said catheter.

4. The device of claim 1 wherein said heating element is coupled to a support member for positioning heating element with respect to the distal end of said catheter.

5. The device of claim 1 wherein said support member is a wire.

6. The device of claim 1, further comprising a power supply coupled to said heating element.

7. The device of claim 1, further comprising a guiding catheter surrounding said catheter.

8. The device of claim 1, further comprising a balloon catheter having an expandable balloon portion, said balloon catheter is disposed coaxially within said catheter, wherein said heating element is coupled around said balloon portion of said balloon catheter.

9. The device of claim 1 wherein said heating element comprises a waveguide having a distal tip, a beam splitter disposed at the distal tip of said waveguide, and a laser directing a laser beam through said waveguide.

10. The device of claim 7 wherein said waveguide comprises a fiber optic cable.

11. The device of claim 1 wherein said heating element comprises a radiofrequency transmitter, an ultrasound transmitter, or a microwave transmitter.

12. The device of claim 1 wherein said heating element comprises an electrical coil.

13. The device of claim 1 wherein said material comprises a polymer.

14. A device comprising:
a catheter having an open distal end;
a support member disposed parallel to said catheter;
and a means of detaching said open distal end from said catheter coupled to said support member, wherein said means of detaching said open distal end from said catheter is a mechanical means or a chemical means.

15. The device of claim 14 wherein said mechanical means comprises a loop surrounding said distal end.

16. The device of claim 15 wherein further comprising a noose disposed between said loop and said wire, wherein said noose tightens said loop around said catheter.

17. The device of claim 15 wherein said loop comprises a plurality of cutting elements, wherein said plurality of cutting elements are capable of detaching said distal end from said catheter.

18. The device of claim 14 wherein said support member comprises a wire.

19. The device of claim 14, further comprising an auxiliary catheter parallel to said catheter, wherein said support member is disposed coaxially within said auxiliary catheter.

20. The device of claim 14 wherein said support member is disposed coaxially within said catheter, and wherein said mechanical means comprises an inner cutting element, said inner cutting element is capable of detaching said distal end from within said catheter.

21. The device of claim 20 wherein said inner cutting element comprises an expandable disk.

22. The device of claim 14 wherein said support member comprises a hollow body and said chemical means comprises a plurality of openings for ejecting a chemical substance from said hollow body, wherein said plurality of openings are disposed circumferentially around the distal end of said hollow body.

23. A method comprising:
a) inserting a catheter having an open distal end;
b) injecting a hardening material through the open distal end to form an obstruction;
c) positioning a heating element proximate the open distal end;
d) heating at least a portion of the open distal end using the heating element so as to detach the catheter from the open distal end and the obstruction.

24. The method of claim 23 wherein the hardening material comprises cyanoacrylate glue, acrylic glue, fibrin glue, adhesives, hydrogels, polymers, or combinations thereof.

25. The method of claim 23 wherein d) comprise inserting heating element coaxially within the catheter.

26. The method of claim 23 wherein d) comprises inserting heating element over the catheter.

27. The method of claim 23 wherein e) comprises applying power to the heating element.

28. The method of claim 23 wherein the heating element comprises a resistance coil, a radiofrequency generator, an ultrasound transmitter, a microwave transmitter, or combinations thereof.

29. The method of claim 23 wherein said heating element comprises a waveguide having a distal tip, a beam splitter disposed at the distal tip of said waveguide, and a laser emitting a laser beam.

30. The method of claim 29 wherein e) comprises directing the laser beam from the laser through the waveguide to the beam splitter so as to form a plurality of beams directed at the catheter and cause the catheter to detach from the open distal end and the obstruction.

31. A method comprising:
a) inserting a catheter having an open distal end;
b) injecting a hardening material through the open distal end to form an obstruction;
c) positioning a means for detaching the catheter from the obstruction at the distal end, wherein said means for detaching the catheter from the obstruction is a mechanical means or a chemical means;
d) using the means for detaching the catheter from the obstruction to detach the catheter from the obstruction.

32. The method of claim 31 wherein the mechanical means comprises a wire and a loop surrounding said distal end, wherein said loop is coupled to said wire.

33. The method of claim 32 wherein d) comprises pushing the loop against the obstruction to force catheter away from the obstruction.

34. The method of claim 32 wherein the mechanical means further comprises a noose disposed between said loop and said wire, wherein said noose tightens said loop around said catheter.

35. The method of claim 32 wherein said loop comprises a plurality of cutting elements, wherein said plurality of cutting elements are capable of severing said distal end from said catheter.

36. The method of claim 31 wherein c) comprise inserting the means for detaching the catheter from the obstruction coaxially within the catheter.

37. The method of claim 36 wherein the mechanical means comprises a support member coupled to an expandable inner cutting element.

38. The method of claim 36 wherein c) comprises inserting the expandable cutting element in a collapsed position and expanding the inner cutting element.

39. The method of claim 36 wherein d) comprises cutting the catheter from within the catheter using the expandable inner catheter element to detach the catheter from the obstruction.

40. The method of claim 31 wherein c) comprises inserting the mechanical means over the catheter.

41. The method of claim 31 wherein the chemical means comprises a hollow body disposed coaxially within said catheter, the hollow body having a plurality of openings disposed circumferentially around the distal end of the hollow body.

42. The method of claim 41 wherein d) comprises injecting a chemical substance through the hollow body, and ejecting the chemical substance through the plurality of openings to dissolve a portion of the catheter and detach the catheter from the obstruction.

43. The method of claim 41 wherein the chemical substance comprises a solvent, an acid, or combinations thereof.

44. The method of claim 41 wherein the hardening material comprises cyanoacrylate glue, acrylic glue, fibrin glue, adhesives, hydrogels, polymers, or combinations thereof.
